(19) United States
(12) Patent Application Publication
(10) Pub. No.: US 2022/0040071 A1
(43) Pub. Date: Feb. 10, 2022

(54) COSMETIC COMPOSITION FOR KERATIN FIBERS COMPRISING C14-20 FATTY ACID AND SEMICRYSTALLINE POLYMER

(71) Applicant: L'OREAL, Paris (FR)

(72) Inventors: Mohamed BOULARAS, Chevilly Larue (FR); Philippe ILEKTI, Chevilly Larue (FR); Maitena LEURIDAN, Chevilly Larue (FR)

(73) Assignee: L'OREAL, Paris (FR)

(21) Appl. No.: 17/416,145

(22) PCT Filed: Dec. 20, 2019
(86) PCT No.: PCT/EP2019/086693

§ 371 Date: Jun. 18, 2021

(30) Foreign Application Priority Data
Dec. 21, 2018 (FR) 1873835

(51) Int. Cl. A61K 8/37; A61K 8/34; A61K 8/81; A61Q 1/10; A61K 8/06

(52) U.S. Cl. CPC A61K 8/375; A61K 8/37; A61K 8/342; A61K 2800/432; A61K 8/8176; A61Q 1/10; A61K 8/06; A61K 8/8147

(57) ABSTRACT

The present invention relates to a composition comprising: (1) at least one ester chosen from: (i) the linear fatty acid monoesters of formula (I) R₁—O—R₂ in which R₁ and R₂ are linear and saturated and have, independently of each other, a number of carbon atoms greater than or equal to 20, with R₁ representing an acyl radical and R₂ representing an alkyl radical, and (ii) fatty acid esters of polyol, said fatty acid(s) having a number of carbon atoms greater than or equal to 20, (2) at least one fatty acid having from 14 carbon atoms to less than 20 carbon atoms, (3) at least one base that is capable of at least partially neutralizing said fatty acid (2), (4) at least one fatty alcohol, (5) at least one semicrystalline polymer, (6) at least one nonvolatile hydrocarbon-based oil, and (7) water.

The present invention relates to the field of caring for and/or making up keratin materials, and is directed towards proposing compositions more particularly intended for making up the eyelashes or the eyebrows.

The term “keratin materials” preferably means human keratin materials, notably keratin fibers.

The term “keratin fibers” in particular means the eyelashes and/or the eyebrows, and preferably the eyelashes. For the purposes of the present invention, this term “keratin fibers” also extends to synthetic false eyelashes.

### TECHNICAL FIELD

In general, compositions intended for making up keratin fibers, for example the eyelashes, aim to densify the thickness and the visual perception of the eyelashes and ultimately the gaze. These mascaras are described as aqueous or cream mascaras when they are formulated in an aqueous base, and anhydrous mascaras when they are formulated as a dispersion in an organic solvent medium.

A great diversity of cosmetic effects may be afforded by applying a mascara to keratin fibers and notably the eyelashes, for instance a volumizing, lengthening, thickening and more particularly charging makeup effect.

These effects are mostly adjusted through the amount and nature of the particles and most particularly those of the waxes present in the mascaras.

In general, mascaras in fact have a significant amount of wax(es) and notably from 10% to 35% by weight of waxes, more generally from 15% to 30% by weight, relative to the total weight thereof.

For obvious reasons, improving the textures of mascara which condition the manifestation of one or more makeup effects is a constant preoccupation of cosmetic formulators.

Moreover, the specific effects associated with a particular formulation, for example charging and moreover providing excellent separation of the made-up eyelashes, are expected to be reproduced virtually identically by all the production batches of one and the same formulation.

In order to meet these expectations and/or objectives, it is therefore necessary to be capable of precisely adjusting the texture of a mascara and of reproducing it as faithfully as possible with batches that are not necessarily produced at the same time but which are identical in terms of ingredients and must therefore provide makeup effects that are in theory also identical.

### PRIOR ART

However, as specified above, most of the mascaras currently available are formulated with a significant amount of waxes. In point of fact, as detailed in Ullmann's Encyclopedia of Industrial Chemistry 2015, Wiley-VCH Verlag GmbH & Co. KGaA, Weinheim 10.1002/14356007.a28.pub2, most waxes are not constituted of a single chemical compound, but are instead complex mixtures. They may be mixtures of oligomers and/or of polymers which, in many cases, also have varied molar masses, varied molar mass distributions and also varied degrees of branching. Thus, a polar wax is conventionally made up of a mixture of alkanes, fatty alcohols and fatty esters, the fatty-chain length of which varies according to the melting point.

It is therefore very difficult for the producers of these waxes to guarantee a rigorously identical composition for all production batches. More specifically, there may be, between several production batches of one and the same wax, a variability in terms of the chemical nature of some of its constituent compounds. Likewise, the proportionality of some of its constituent compounds may also vary between production batches.

For obvious reasons, these variabilities have a not insignificant impact on the properties of the wax and therefore on those of the mascara incorporating this wax in significant amount. Thus, two mascara formulations of identical composition and therefore produced from one and the same conventional wax and in the same amount, can nevertheless diverge in terms of rheological properties and therefore of texture, if they were produced from two separate production batches of this wax.

### PRESENTATION OF THE INVENTION

Consequently, the use of conventional waxes, in particular in significant amount, in mascara compositions does not make it possible to guarantee for users the reproduction of finely adjusted and totally identical rheological properties in all the mascara specimens of one and the same composition.

The present invention aims precisely to provide mascara compositions which have overcome this limitation.

Thus, a first objective of the present invention is to obtain mascara compositions whose texturing properties are finely adjustable and reproducible.

Another objective of the present invention is to provide a mascara architecture produced using a predominant weight proportion of single-component ingredients. The predominant use of single-component ingredients advantageously makes it possible to eliminate the risk of composition variability that may exist between several production batches of a multicomponent ingredient and therefore to eliminate the risk of its impact on the final properties of the mascara.

Another objective of the present invention is to provide a mascara architecture which makes it possible to significantly reduce or even dispense with the use of waxes, but which nevertheless remains very satisfactory in terms of makeup effect.

Thus, another objective of the invention is to provide consumers with compositions, notably cosmetic compositions, and in particular mascaras, that are compatible with a long playtime while at the same time making it possible to preserve the separation of the eyelashes.

Another objective of the invention is to also provide consumers with compositions which have a creamy texture.

Finally, an objective of the invention is to meet, for the most part, the abovementioned objectives while at the same time providing the composition with great stability.

The term “stable composition” means that the composition remains usable as makeup after storage for two months at a temperature of 45° C. and that it retains its pleasantness and its sensory signature on application. More specifically, a “stable composition” according to the invention has an acceptable change in viscosity, that is to say that the difference between the initial viscosity and the viscosity after storage for two months at a temperature of 45° C. remains less than 10 Pa·s.

Unexpectedly, the inventors have found that it is possible to significantly or even totally dispense with waxes as texturing agent in formulations for making up and/or caring for keratin fibers and therefore with the limitations mentioned above, on condition that very specific compounds are combined in such formulations.

### SUMMARY OF THE INVENTION

Thus, according to a first of its aspects, the present invention relates to a composition, preferably a cosmetic composition for caring for and/or making up keratin materials, in particular the eyelashes and/or the eyebrows, comprising:
(1) at least one ester chosen from:
(i) linear fatty acid monoesters of formula (I):

[Chem 1]

R₁—O—R₂ (I)

in which R₁ and R₂ are linear and saturated and have, independently of one another, a number of carbon atoms greater than or equal to 20, with R₁ representing an acyl radical, and R₂ representing an alkyl radical, and
(ii) fatty acid esters of polyol, said fatty acid(s) having a number of carbon atoms greater than or equal to 20,
(2) at least one fatty acid having from 14 carbon atoms to less than 20 carbon atoms,
(3) at least one base that is capable of at least partially neutralizing said fatty acid (2),
(4) at least one fatty alcohol preferably chosen from C₁₄-C₃₀ fatty alcohols, better still chosen from the linear and saturated C₁₄-C₂₄ and better still C₁₄-C₂₀ fatty alcohols,
(5) at least one semicrystalline polymer,
(6) at least one nonvolatile hydrocarbon-based oil, and
(7) water.

Unexpectedly, the inventors have in fact found that the formulation in an aqueous medium of at least one ester chosen from the linear fatty acid monoesters of formula (I) (i) and the fatty acid esters of polyol (ii), in combination with at least one neutralized form of a fatty acid having from 14 carbon atoms to less than 20 carbon atoms, at least one fatty alcohol (4), at least one semicrystalline polymer (5) and at least one nonvolatile hydrocarbon-based oil (6) affords access to compositions whose texture can be finely adjusted and guaranteed in terms of reproducibility.

As emerges from what follows, these new compositions are advantageous in several respects.

First of all, compounds (1) to (6) required according to the invention are of synthetic origin and, in this respect, of increased purity when compared, for example, with a natural wax.

They are also, as individualized compound, single-component compound or compound with a well-defined number of components, as opposed to the majority of conventional waxes which are often multicomponent or even have an indefinite number of compounds, such as natural waxes and some synthetic waxes.

These two specificities are particularly advantageous since they make it possible to eliminate a risk of variability with regard to their respective compositions.

As emerges from the examples below, the compositions in accordance with the invention and based on the use of compounds (1) to (6) as texturing agent prove to be very satisfactory in terms of makeup effects.

Thus, compositions according to the invention may have a creamy texture which proves to be finely adjustable by virtue of the use of the required combination according to the invention.

The obtention of these properties is conditioned by the use of the compounds (1) to (6) and advantageously does not therefore require the additional presence of waxes, in particular in significant amount.

Thus, the compositions according to the invention advantageously comprise less than 5% of waxes as defined below.

The term “waxes” refers to lipophilic compounds, which are solid at room temperature (20° C.) and at atmospheric pressure (760 mmHg), with a reversible solid/liquid change of state, which have a melting point of greater than or equal to 40° C., which may be up to 120° C.

For the purposes of the invention, the waxes to which this abovementioned amount limitation relates are distinct from those capable of being embodied by the component which is a fatty acid ester of polyol (1) and fatty alcohol (4) required according to the invention.

Finally, the manifestation of the makeup effects afforded by the combination of compounds (1) to (6) is not acquired to the detriment of the stability of the compositions.

According to another of its aspects, the present invention relates to a process, notably a cosmetic process for caring for and/or making up keratin materials, in particular the eyelashes and/or the eyebrows, comprising at least one step consisting in applying, to said keratin materials, in particular the eyelashes and/or the eyebrows, a composition in accordance with the invention.

### DETAILED DESCRIPTION

Ester (1)

As stated above, a composition according to the invention comprises at least one ester (1) chosen from:
(i) linear fatty acid monoesters of formula (I):

[Chem 2]

R₁—O—R₂ (I)

in which R₁ and R₂ are linear and saturated and have, independently of each other, a number of carbon atoms greater than or equal to 20, with R₁ representing an acyl radical, and R₂ representing an alkyl radical, and
(ii) fatty acid esters of polyol, said fatty acid(s) having a number of carbon atoms greater than or equal to 20.

The composition according to the invention may comprise at least 5.0% by weight, preferably at least 6.0% by weight, better still at least 7.0% by weight of ester (1) relative to the total weight of the composition.

According to a particularly preferred embodiment of the invention, the ester(s) (1) are present in the composition in a content ranging from 6.0% to 35.0% by weight, preferably from 7.0% to 30.0%, or even preferably from 8.0% to 28.0% by weight, relative to the total weight of the composition.

According to a first variant of the invention, an ester (1) is a linear fatty acid monoester (i) which corresponds to formula (I) below:

[Chem 3]

R₁—O—R₂ (I)

in which R₁ and R₂ are linear and saturated and have, independently of each other, a number of carbon atoms greater than or equal to 20, with R₁ representing an acyl radical, and R₂ representing an alkyl radical.

This or these fatty acid monoester(s) are used during the preparation of a composition according to the invention, in an individualized form or in the form of a mixture exclusively comprising linear fatty acid monoesters of formula (I).

In a preferred embodiment, the fatty acid monoester(s) have a melting point greater than 50° C.

The melting point may be measured by any known method and in particular using a differential scanning calorimeter (DSC).

According to a preferred embodiment of the invention, the acyl and alkyl radicals representing R₁ and R₂, respectively, are chosen in such a way that the compound of formula (I) is solid at a temperature of less than or equal to 30° C.

According to a particularly preferred embodiment, R₁ and R₂ are, respectively, acyl and alkyl radicals having the same number of carbon atoms.

Thus, according to a preferred embodiment, the ester (1) is at least one linear fatty acid monoester of formula (I) in which R₁ and R₂ are, respectively, acyl and alkyl radicals having the same number of carbon atoms. Preferably, R₁ and R₂ are, respectively, acyl and alkyl radicals having a number of carbon atoms ranging from 20 to 30, preferably from 20 to 24.

In particular, the fatty acid monoester according to the invention is chosen from arachidyl arachi date and behenyl behenate.

According to a particularly preferred embodiment of the invention, the linear fatty acid monoester is a behenyl behenate.

A behenyl behenate that is suitable for use in the composition according to the invention may in particular be Kester Wax K-72 sold by the company Koster Keunen, Dub BB sold by the company Stearinerie Dubois, or Dermowax BB sold by Alzo.

According to a second variant of the invention, an ester (1) is at least one fatty acid ester of polyol (ii), said fatty acid(s) having a number of carbon atoms greater than or equal to 20.

For the purposes of the invention, the term “fatty acid ester of polyol” comprises both fatty acid monoesters and polyesters of polyol.

The composition according to the invention may comprise a single fatty acid ester of polyol (ii) or several fatty acid esters of polyol (ii).

If several esters are present, they may be added separately during the preparation of the composition and the mixture thereof can then be formed in situ. They may also be used in the form of a mixture which is already commercially available and in which the weight proportion and the degree of purity of each of the esters are controlled. In other words, the composition of these mixtures is faithfully reproducible, as opposed to mixtures of fatty acid esters of polyol generated via an esterification process in which the fatty-chain distribution of the initial reagents results in a mixture of several ester compounds of complex composition; for instance KesterWax K82P appearing under the INCI name Synthetic Beeswax.

In particular, the fatty acid ester of polyol is chosen from fatty acid esters of glycerol.

According to a preferred embodiment of the invention, the ester (1) is at least one fatty acid ester of polyol (ii) chosen from the fatty acid esters of glycerol of formula (II) below:

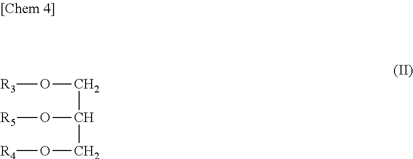

in which R₃, R₄ and R₅ may represent, independently of each other, a hydrogen atom H or a saturated or unsaturated, linear or branched acyl radical having a number of carbon atoms greater than or equal to 20, at least one from among R₃, R₄ and/or R₅ representing a saturated or unsaturated, linear or branched acyl radical having a number of carbon atoms greater than or equal to 20.

According to a preferred embodiment of the invention, the acyl radicals representing R₃, R₄ and R₅, respectively, are chosen in such a way that the compound of formula (II) is solid at a temperature of less than or equal to 30° C.

In a preferred embodiment, the fatty acid esters of glycerol according to the invention have a melting point of greater than 50° C.

The melting point may be measured by any known method and in particular using a differential scanning calorimeter (DSC).

In a preferred embodiment of the invention, when R₃, R₄ and/or R₅ represent, independently of each other, acyl radicals, these acyl radicals are linear. In a preferred embodiment of the invention, when R₃, R₄ and/or R₅ represent, independently of each other, acyl radicals, these acyl radicals are saturated. According to a particularly preferred embodiment of the invention, when R₃, R₄ and/or R₅ represent, independently of each other, acyl radicals, these acyl radicals are linear and saturated.

In a preferred embodiment of the invention, when R₃, R₄ and/or R₅ represent, independently of each other, acyl radicals, these acyl radicals are identical.

According to a preferred embodiment of the invention, when R₃, R₄ and/or R₅ represent, independently of each other, acyl radicals, these radicals have a number of carbon atoms ranging from 20 to 30, preferably from 20 to 24. In particular, R₃, R₄ and/or R₅ are chosen from arachidyl and behenyl, better still behenyl.

Preferably, when R₃, R₄ and/or R₅ represent, independently of each other, acyl radicals, these radicals are behenyl radicals.

According to a particularly preferred embodiment of the invention, the fatty acid ester(s) of polyol (ii) are chosen from a fatty acid tri ester of glycerol and a mixture of fatty acid monoester, diester and triester of glycerol.

In particular, the fatty acid ester(s) of polyol (ii) are chosen from the behenic acid triester of glycerol (or tribehenin) and a mixture of behenic acid monoesters, diesters and triesters of glycerol, and better still is tribehenin.

The behenic acid monoesters, diesters and triesters of glycerol are also found under the names glyceryl behenate, glyceryl dibehenate and glyceryl tribehenate (or tribehenin), respectively.

A behenic acid triester of glycerol that may be suitable for use in the invention is, for example, the product sold under the name Synchrowax HRC-PA-(MH) sold by the company Croda.

A mixture of behenic acid monoesters, diesters and triesters of glycerol that may be suitable for use in the invention is, for example, Compritol 888 CG ATO sold by the company Gattefosse.

Ionic Surfactant

As specified above, the ester(s) (1) used according to the invention are combined with at least one ionic surfactant resulting from the neutralization of a fatty acid (2) including from 14 to less than 20 carbon atoms with a base (3).

More specifically, the ionic surfactant under consideration according to the invention results from the at least partial neutralization of the carboxylic functions of a fatty acid (2) comprising from 14 to less than 20 carbon atoms with a base (3).

Fatty Acid (2)

The fatty acid according to the invention comprises from 14 to less than 20 carbon atoms. According to one preferred embodiment of the invention, the fatty acid comprises from 16 to less than 20 carbon atoms. According to a particularly preferred embodiment, the number of carbon atoms of the fatty acid ranges from 16 to 18.

In particular, the fatty acid(s) according to the invention are chosen from linear fatty acids, saturated fatty acids and mixtures thereof.

According to a particularly advantageous embodiment of the invention, the fatty acid of the ionic surfactant is chosen from linear and saturated fatty acids.

According to a particular embodiment of the invention, the fatty acid(s) are chosen from palmitic acid, stearic acid and mixtures thereof, and preferably comprise at least stearic acid having the INCI name STEARIC ACID.

According to a particularly preferred embodiment of the invention, the composition uses, as fatty acid (2), a mixture of C₁₆-C₁₈ fatty acids, preferably a mixture of fatty acids having 16 carbon atoms, such as palmitic acid, and of fatty acids having 18 carbon atoms, such as stearic acid.

A preferred stearic acid that is suitable for use in the invention is, for example, Stearic Acid 1850 sold by the company Southern Acids.

A composition according to the invention may comprise at least 3.0% by weight of fatty acid(s) (2), in particular stearic acid, relative to the total weight of the composition.

According to a preferred embodiment of the invention, the fatty acid is present in a content ranging from 3.5% to 20.0% by weight, preferably from 4.0% to 20.0% by weight, better still from 4.5% to 15.0% by weight, even better still from 5.0% to 15.0% by weight of fatty acid (2), in particular of stearic acid, relative to the total weight of the composition.

As mentioned above, this fatty acid is used in an ionic form generated via its interaction with an inorganic base. This base is used at an amount sufficient to be capable of at least partially neutralizing at least one fatty acid (2).

Base (3)

The composition according to the invention comprises at least one base. This base may be organic or inorganic.

According to a first variant, the base is at least one organic base.

This organic base may be chosen from ammonium, and amine and amino alcohol derivatives thereof.

Preferably, the base of organic origin is chosen from alkanolamine derivatives such as monoethanolamine, diethanolamine, triethanolamine, triisopropanolamine, aminomethylpropanol, tromethamine and aminomethylpropanediol, and mixtures thereof. According to a particular embodiment, the base is a primary (poly)hydroxyalkylamine. The term “primary (poly)hydroxyalkylamine” in particular means a primary dihydroxyalkylamine, it being understood that the term “primary” refers to a primary amine function, i.e. —NH₂, and the alkyl group being a linear or branched C₁-C₈, preferably branched C₄, hydrocarbon-based chain, such as 1,3-dihydroxy-2-methylpropyl. The primary (poly)hydroxyalkylamine is preferably 1,3-dihydroxy-2-methyl-2-propylamine (also known as aminomethylpropanediol or AMPD).

According to a preferred embodiment of the invention, the base of organic origin is chosen from alkanolamine derivatives, notably chosen from triethanolamine, aminomethylpropanol, aminomethylpropanediol, and mixtures thereof, and is preferably at least aminomethylpropanediol.

Such an aminomethylpropanediol that is suitable for use in the invention is, for example, AMPD Ultra PC sold by the company Angus (Dow Corning).

According to a second variant, the base is at least one inorganic base.

This inorganic base may be chosen from alkali metal hydroxides.

Preferably, the inorganic base is chosen from sodium hydroxide and potassium hydroxide.

According to a preferred embodiment of the invention, the inorganic base is sodium hydroxide.

The amount of base is adjusted so as to obtain sufficient neutralization to confer effective ionicity on the associated fatty acid (2).

Preferably, the base is present in an amount sufficient to neutralize some or all of the carboxylic functions of the fatty acid(s) (2) comprising from 14 to less than 20 carbon atoms. In a preferred embodiment, the amount of base is such that it is capable of neutralizing all the acid functions of the fatty acid (2).

According to a preferred embodiment, the composition according to the invention comprises at least 0.1% by weight, better still at least 0.15% by weight, of base(s) relative to the total weight of the composition.

According to a preferred embodiment of the invention, the base is present in a content ranging from 0.2% to 3.0% by weight, preferably from 0.3% to 2.0% by weight of base(s) relative to the total weight of the composition.

In particular, bases that are suitable for use according to the invention are sodium hydroxide or aminomethylpropanediol.

According to a particular embodiment of the invention, the compositions according to the invention contain an ionic surfactant derived from the total neutralization of stearic acid with sodium hydroxide.

According to another particular embodiment of the invention, the compositions according to the invention contain an ionic surfactant derived from the total neutralization of stearic acid with aminomethylpropanediol.

The fatty acid (2) and the base (3) making up the neutralized ionic surfactant according to the invention may be introduced into the composition in the form of one and the same commercial material, or one after the other in the form of two distinct commercial materials. Preferably, the fatty acid (2) and the base (3) will be introduced into the composition in the form of two distinct commercial materials.

Fatty Alcohol (4)

The compositions according to the invention also comprise at least one fatty alcohol. A composition can therefore comprise a single fatty alcohol according to the invention or several distinct fatty alcohols.

If several distinct fatty alcohols are present, they can be added separately during the preparation of the composition and the mixture thereof can then be formed in situ. They can also be used in the form of a mixture which is already commercially available and in which the weight proportion and the degree of purity of each of the fatty alcohols are controlled. In other words, the composition of these mixtures is faithfully reproducible, as opposed to mixtures of fatty alcohols generated via synthesis from starting materials derived from complex mixtures.

The fatty alcohol(s) are in particular chosen from linear or branched, saturated or unsaturated C₁₄-C₃₀, preferably C₁₄-C₂₄, and better still C₁₄-C₂₀, fatty alcohols.

The fatty alcohol(s) are in particular chosen from linear and saturated C₁₄-C₃₀ fatty alcohols, preferably linear and saturated C₁₄-C₂₄ fatty alcohols, and better still linear and saturated C₁₄-C₂₀ fatty alcohols.

According to a particularly preferred embodiment, the fatty alcohol is in the form of a mixture of several different fatty alcohols, and is preferably a mixture of several linear and saturated C₁₄-C₃₀, better still C₁₄-C₂₄, even better still C₁₄-C₂₀, fatty alcohols.

Preferably, the fatty alcohol according to the invention is chosen from cetyl alcohol (C₁₆), stearyl alcohol (C₁₈) and mixtures thereof (also known as “cetearyl alcohol”). Preferentially, the fatty alcohol according to the invention is a mixture of cetyl alcohol and stearyl alcohol. Such a mixture is notably sold under the name Lannette O OR/MB by the company BASF.

According to a preferred embodiment, the fatty alcohol is solid at room temperature.

In particular, a composition according to the invention comprises at least from 1.0% to 20.0% by weight, preferably from 2.0% to 15.0% by weight and even more particularly from 3.0% to 10.0% by weight of fatty alcohol(s) relative to the total weight of the composition.

Semicrystalline Polymer (5)

The composition according to the invention comprises at least 2.0% by weight, preferably from 3.0% to 20.0% by weight, better still from 4.0% to 15.0% by weight, or even from 5.0% to 15.0% by total weight of semicrystalline polymer(s), relative to the total weight of the composition.

For the purposes of the invention, the term “polymers” means compounds including at least two repeating units, preferably at least three repeating units and more especially at least ten repeating units.

For the purposes of the invention, the term “semicrystalline polymer” means polymers including a crystallizable portion and an amorphous portion and having a first-order reversible change of phase temperature, in particular of melting (solid-liquid transition). The crystallizable portion is preferably a chain that is lateral (or a chain that is pendent) relative to the backbone.

Besides the crystallizable chains or blocks, the blocks of the polymers are amorphous.

For the purposes of the invention, the term “crystallizable chain or block” means a chain or block which, if it were alone, would change from the amorphous state to the crystalline state reversibly, depending on whether the temperature is above or below the melting point. For the purposes of the invention, a chain is a group of atoms, which is pendent or lateral relative to the polymer backbone.

When the crystallizable portion is a chain that is pendent relative to the backbone, the semicrystalline polymer may be a homopolymer or a copolymer.

Preferably, the semicrystalline polymer has an organic structure.

The term “organic compound” or “having an organic structure” refers to compounds containing carbon atoms and hydrogen atoms and optionally heteroatoms such as S, O, N or P, alone or in combination.

The semicrystalline polymer(s) according to the invention are solids at room temperature (25° C.) and atmospheric pressure (760 mmHg), the melting point of which is greater than or equal to 30° C.

The melting point values correspond to the melting point measured using a differential scanning calorimeter (D.S.C.) such as the calorimeter sold under the name DSC 30 by the company Mettler, with a temperature rise of 5° C. or 10° C. per minute (the melting point considered is the point corresponding to the temperature of the most endothermic peak in the thermogram).

According to a particular embodiment, the semicrystalline polymer(s) used in the composition of the invention have a melting point m.p. of less than 95° C., preferably less than 85° C. The semicrystalline polymer(s) may thus have a melting point m.p. ranging from 30° C. to 95° C. and preferably from 40° C. to 85° C. This melting point is preferably a first-order change of state temperature.

According to the invention, the semicrystalline polymers are advantageously soluble in the fatty phase, notably to at least 1.0% by weight, at a temperature above their melting point. Preferably, the polymer backbone of the semicrystalline polymers is soluble in the fatty phase at a temperature above their melting point.

Preferably, the crystallizable blocks or chains of the semicrystalline polymers represent at least 30% of the total weight of each polymer and better still at least 40%.

When the semicrystalline polymers of the invention are crystallizable block polymers, they may be block or multiblock copolymers. They may be obtained by polymerizing a monomer bearing reactive (or ethylenic) double bonds or by polycondensation. When the polymers of the invention are polymers bearing crystallizable side chains, these polymers are advantageously in random or statistical form.

Preferably, the semicrystalline polymers of the invention are of synthetic origin.

According to a preferred embodiment, the semicrystalline polymer is chosen from homopolymers and copolymers including units resulting from the polymerization of one or more monomers bearing crystallizable hydrophobic side chain(s).

The semicrystalline polymers that may be used in the invention may be chosen in particular from homopolymers or copolymers, in particular those bearing at least one crystallizable side chain, such as those described in U.S. Pat. No. 5,156,911.

In a preferred embodiment, the crystallizable side chain(s) are hydrophobic.

These homopolymers or copolymers may result:

from the polymerization, notably radical polymerization, of one or more monomers bearing double bond(s) that are reactive or ethylenic with respect to a polymerization, namely bearing a vinyl, (meth)acrylic or allylic group,

from the polycondensation of one or more monomers bearing co-reactive groups (carboxylic acid, sulfonic acid, alcohol, amine or isocyanate), for instance polyesters, polyurethanes, polyethers or polyureas.

In general, the crystallizable units (chains or blocks) of the semicrystalline polymers according to the invention are derived from monomer(s) containing crystallizable block(s) or chain(s), used for manufacturing semicrystalline polymers. These polymers are preferably chosen notably from homopolymers and copolymers resulting from the polymerization of at least one monomer containing crystallizable chain(s) that may be represented by formula A below:

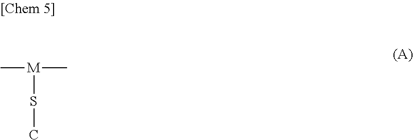

with M representing an atom of the polymer backbone, C representing a crystallizable group, and S representing a spacer, the crystallizable “—S—C” chains being hydrocarbon-based aliphatic or aromatic chains, including saturated or unsaturated hydrocarbon-based alkyl chains, which are for example C₁₀-C₄₀, preferably C₁₀-C₃₀. “C” notably represents a linear or branched or cyclic group (CH₂)ₙ, with n being an integer ranging from 10 to 40. Preferably, “C” is a linear group. Preferably, “S” and “C” are different.

When the crystallizable chains are hydrocarbon-based aliphatic chains, they include hydrocarbon-based alkyl chains containing at least 10 carbon atoms and not more than 40 carbon atoms and better still not more than 30 carbon atoms. They are notably aliphatic chains or alkyl chains containing at least 10 carbon atoms, and they are preferably C₁₀-C₄₀, preferably C₁₀-C₃₀, alkyl chains.

Preferably, the crystallizable chains are C₁₀-C₃₀ hydrocarbon-based aliphatic chains.

As examples of semicrystalline homopolymers or copolymers containing crystallizable chain(s) that are suitable for use in the invention, mention may be made of those resulting from the polymerization of one or more of the following monomers: saturated alkyl (meth)acrylates with the alkyl group being C₁₀-C₃₀, N-alkyl(meth)acrylamides with the alkyl group being C₁₀ to C₃₀, vinyl esters containing alkyl chains with the alkyl group being C₁₀ to C₃₀, vinyl ethers containing alkyl chains with the alkyl group being C₁₀ to C₃₀, C₁₀ to C₃₀ alpha-olefins, for instance octadecene, para-alkylstyrenes with an alkyl group including from 10 to 30 carbon atoms, and mixtures thereof.

When the polymers result from a polycondensation, the hydrocarbon-based crystallizable chains as defined above are borne by a monomer that may be a diacid, a diol, a diamine or a diisocyanate.

When the polymers that are subjects of the invention are copolymers, they additionally contain from 0% to 50% of groups Y which is a polar monomer, a nonpolar monomer or a mixture of the two.

When Y is a polar monomer, it is either a monomer bearing polyoxyalkylene groups (notably oxyethylene and/or oxypropylene groups), a hydroxyalkyl (meth)acrylate, for instance hydroxyethyl acrylate, (meth)acrylamide, an N-alkyl(meth)acrylamide, an N,N-dialkyl(meth)acrylamide, for instance N,N-diisopropylacrylamide or N-vinylpyrrolidone (NVP), N-vinylcaprolactam, a monomer bearing at least one carboxylic acid group, for instance (meth)acrylic acid, crotonic acid, itaconic acid, maleic acid or fumaric acid, or bearing a carboxylic acid anhydride group, for instance maleic anhydride, and mixtures thereof.

When Y is a nonpolar monomer, it may be an ester of the linear, branched or cyclic alkyl (meth)acrylate type, a vinyl ester, an alkyl vinyl ether, an α-olefin, styrene or styrene substituted with a C₁ to C₁₀ alkyl group, for instance α-methylstyrene, or a macromonomer of the polyorganosiloxane type containing vinyl unsaturation.

Preferably, the semicrystalline polymers containing crystallizable side chain(s) are alkyl (meth)acrylate or alkyl(meth)acrylamide homopolymers with an alkyl group as defined above, and notably of C₁₀-C₃₀, copolymers of these monomers with a hydrophilic monomer preferably of different nature from (meth)acrylic acid, for instance N-vinylpyrrolidone or hydroxyethyl (meth)acrylate, and mixtures thereof.

It is also possible to use the semicrystalline polymers obtained by copolymerization of behenyl acrylate and of acrylic acid or of N-vinylpyrrolidone, as described in U.S. Pat. No. 5,519,063.

Advantageously, the semicrystalline polymer(s) containing crystallizable side chain(s) have a weight-average molecular mass Mw ranging from 5000 g/mol to 1 000 000 g/mol, preferably from 10 000 g/mol to 800 000 g/mol, preferentially from 15 000 g/mol to 500 000 g/mol, and more preferably from 80 000 g/mol to 200 000 g/mol.

According to a particular embodiment of the invention, the semicrystalline polymer may be chosen from homopolymers and copolymers resulting from the polymerization of at least one monomer containing crystallizable side chain(s) chosen from saturated C₁₀ to C₃₀ alkyl (meth)acrylates, which may be represented by the following formula:

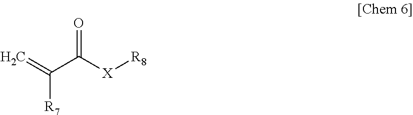

in which R₇ is H or CH₃, R₈ represents a C₁₀ to C₃₀ alkyl group and X represents O.

According to a more particular embodiment of the invention, the semicrystalline polymer results from the polymerization of monomers containing crystallizable side chain(s), chosen from saturated C₁₀ to C₃₀ alkyl (meth)acrylates.

The semicrystalline polymers comprising a crystallizable side chain may be chosen from copolymers resulting from the copolymerization of acrylic acid and C₁₀ to C₃₀ alkyl (meth)acrylate, notably such as those described in patent U.S. Pat. No. 5,156,911.

The semicrystalline polymers may notably be those described in Examples 3, 4, 5, 7 and 9 of patent U.S. Pat. No. 5,156,911, and more particularly those obtained by the copolymerization:

of acrylic acid, of hexadecyl acrylate and of isodecyl acrylate in a 1/16/3 ratio,

of acrylic acid and of pentadecyl acrylate in a 1/19 ratio,

of acrylic acid, of hexadecyl acrylate and of ethyl acrylate in a 2.5/76.5/20 ratio,

of acrylic acid, of hexadecyl acrylate and of methyl acrylate in a 5/85/10 ratio,

of acrylic acid and of polyoctadecyl (meth)acrylate in a 2.5/97.5 ratio.

As a particular example of a semicrystalline polymer that may be used in the composition according to the invention, mention may be made of the Intelimer® products from the company Landec described in the brochure “Intelimer® polymers”, Landec IP22 (Rev. 4-97). These polymers are in solid form at room temperature (25° C.). They bear crystallizable side chains and have the preceding formula (A). They are poly(C₁₀-C₃₀)alkyl acrylates, which are particularly suitable as semicrystalline polymers that may be included in a composition in accordance with the present invention.

According to a particularly preferred embodiment of the invention, the semicrystalline polymer(s) (5) according to the invention are derived from a monomer containing a crystallizable chain chosen from saturated C₁₀ to C₃₀ alkyl (meth)acrylates and more particularly from poly(stearyl acrylate)s, poly(behenyl acrylate)s, and mixtures thereof.

In particular, the semicrystalline polymer(s) (5) are at least one poly(behenyl acrylate).

Preferably, the semicrystalline polymers that are suitable for use in the invention are notably poly(stearyl acrylate), in particular the product sold under the name Intelimer® IPA 13-1, from the company Air Products and Chemicals or Landec, which is a poly(stearyl acrylate) with a melting point equal to 49° C., or poly(behenyl acrylate), sold under the name Intelimer® IPA 13-6, from the company Air Products and Chemicals or Landec, which is a poly(behenyl acrylate) with a melting point equal to 65° C.

According to a particularly preferred embodiment, the semicrystalline polymer is at least one poly(behenyl acrylate).

Examples of homopolymers or copolymers that are suitable for use as semicrystalline polymers for the invention preferably include from 50% to 100% by weight of units resulting from the polymerization of one or more monomers bearing a crystallizable hydrophobic side chain.

Nonvolatile Hydrocarbon-Based Oil (6)

The term “oil” means a water-immiscible nonaqueous compound that is liquid at room temperature (25° C.) and at atmospheric pressure (760 mmHg).

The term “nonvolatile oil” means an oil that remains on keratin materials, at room temperature and atmospheric pressure, for at least several hours and that notably has a vapor pressure of less than 10⁻³ mmHg (0.13 Pa).

For the purposes of the present invention, the term “hydrocarbon-based oil” means an oil composed of carbon, hydrogen and, where appropriate, oxygen atoms. It is in particular free of silicon atoms and thus is different from a silicone oil.

It may be a polar or apolar oil.

Preferably, a composition according to the invention comprises at least one nonvolatile hydrocarbon-based oil with a molar mass (M_{W}) ranging from 650 to 7500 g.mol⁻¹ and preferably between 750 and 7500 g.mol⁻¹. As illustrations of these high molecular weight oils, mention may be made notably of:

lipophilic polymers such as vinylpyrrolidone copolymers such as: the vinylpyrrolidone/1-hexadecene copolymer sold notably by the company ISP under the name Antaron V-216 (M_{W}=7300 g.mol⁻¹),

esters such as:

linear fatty acid esters with a total carbon number ranging from 35 to 70, for instance pentaerythrityl tetrapelargonate (M_{W}=697 g.mol⁻¹),

hydroxylated esters such as polyglycerol-2 triisostearate (M_{W}=965 g.mol⁻¹),

aromatic esters such as tridecyl trimellitate (M_{W}=757 g.mol⁻¹),

esters of C₂₄-C₂₈ branched fatty acids or fatty alcohols such as those described in patent application EP-A-0 955 039, and notably triisoarachidyl citrate (M_{W}=1033.76 g.mol⁻¹), pentaerythrityl tetraisononanoate (M_{W}=697 g.mol⁻¹), glyceryl triisostearate (M_{W}=891 g.mol⁻¹), glyceryl tris(2-decyl)tetradecanoate (M_{W}=1143 g.mol⁻¹), pentaerythrityl tetraisostearate (M_{W}=1202 g.mol⁻¹), polyglyceryl-2 tetraisostearate (M_{W}=1232 g.mol⁻¹) or pentaerythrityl tetrakis(2-decyl)tetradecanoate (M_{W}=1538 g.mol⁻¹),

a polyester resulting from the esterification of at least one triglyceride of hydroxylated carboxylic acid(s) with an aliphatic monocarboxylic acid and with an aliphatic dicarboxylic acid, which is optionally unsaturated, for instance the succinic acid and isostearic acid castor oil sold under the reference Zenigloss by Zenitech,

esters of a diol dimer and of a diacid dimer of general formula (III):

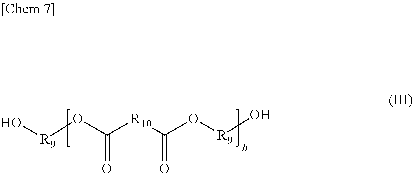

in which:
R₉ represents a diol dimer residue obtained by hydrogenation of dilinoleic diacid,
R₁₀ represents a hydrogenated dilinoleic diacid residue, and
h represents an integer ranging from 1 to 9,
notably the esters of dilinoleic diacids and of dilinoleyl diol dimers sold by the company Nippon Fine Chemical under the trade names Lusplan DD-DA5® and DD-DA7®,
oils of plant origin, such as sesame oil (M_{W}=820 g.mol⁻¹), castor oil,
linear or branched hydrocarbons of synthetic or mineral origin, such as liquid paraffins,
aliphatic hydrocarbons and derivatives thereof,
polydecenes,
polybutenes,
and mixtures thereof.

According to a preferred embodiment, a composition according to the invention comprises between 0.1% and 20% by weight, preferably between 0.5% and 10% by weight and better still between 1% and 8% by weight of nonvolatile oils relative to the total weight of the composition. Preferably, these nonvolatile oils have a molar mass (M_{W}) ranging from 650 to 7500 g.mol⁻¹.

Water (7)

A composition according to the invention comprises water.

In particular, a composition according to the invention comprises at least 30.0% by weight, better still at least 40.0% by weight, or even a content of from 50% to 60% by weight of water, relative to the total weight of the composition.

In one preferred embodiment of the invention, the composition according to the invention includes:

(1) at least behenyl behenate as linear fatty acid monoester,

(2) at least one fatty acid chosen from stearic acid, palmitic acid and mixtures thereof,

(3) at least one base chosen from aminomethylpropanediol and alkali metal hydroxides,

(4) at least one linear and saturated fatty alcohol chosen from those which are C₁₄-C₂₀,

(5) at least one semicrystalline polymer resulting from the polymerization of at least one monomer bearing crystallizable side chain(s),

(6) at least polybutene as nonvolatile high molecular weight oil, and

(7) water.

In another preferred embodiment, the composition according to the invention includes:

(1) at least tribehenin as C₂₀-C₂₄ fatty acid triester of glycerol,

(2) at least one fatty acid chosen from stearic acid, palmitic acid and mixtures thereof,

(3) at least one base chosen from aminomethylpropanediol and alkali metal hydroxides,

(4) at least one linear and saturated fatty alcohol chosen from those which are C₁₄-C₂₀,

(5) at least one semicrystalline polymer resulting from the polymerization of at least one monomer bearing crystallizable side chain(s),

(6) at least polybutene as nonvolatile high molecular weight oil, and

(7) water.

In yet another particularly preferred embodiment, the composition according to the invention includes:

(1) at least behenyl behenate as linear fatty acid monoester,

(2) at least one fatty acid chosen from stearic acid, palmitic acid and mixtures thereof,

(3) at least one base chosen from aminomethylpropanediol and alkali metal hydroxides,

(4) at least one linear and saturated fatty alcohol chosen from those which are C₁₄-C₂₀,

(5) at least one semicrystalline polymer resulting from the polymerization of at least one monomer bearing crystallizable side chain(s),

(6) at least one acid triglyceride as nonvolatile high molecular weight oil, and

(7) water.

In yet another particularly preferred embodiment, the composition according to the invention includes:

(1) at least tribehenin as C₂₀-C₂₄ fatty acid triester of glycerol,

(2) at least one fatty acid chosen from stearic acid, palmitic acid and mixtures thereof,

(3) at least one base chosen from aminomethylpropanediol and alkali metal hydroxides,

(4) at least one linear and saturated fatty alcohol chosen from those which are C₁₄-C₂₀,

(5) at least one semicrystalline polymer resulting from the polymerization of at least one monomer bearing crystallizable side chain(s),

(6) at least one acid triglyceride as nonvolatile high molecular weight oil, and

(7) water.

Other Components

In addition to the abovementioned compounds, a composition according to the invention may of course comprise secondary ingredients.

a) Waxes

Thus, a composition according to the invention may also comprise a wax.

However, with regard to the objectives targeted by the present invention, the compositions according to the invention preferably comprise a reduced amount of wax and notably contain less than 5% by weight and preferably less than 3% by weight of waxes, relative to the total weight of said composition.

As specified in the preamble, for the purposes of the invention, the term “waxes” refers to lipophilic compounds, which are solid at room temperature (20° C.) and at atmospheric pressure (760 mmHg), with a reversible solid/liquid change of state, which have a melting point of greater than or equal to 40° C., which may be up to 120° C.

It is recalled that, for the purposes of the invention, the waxes to which the abovementioned amount limitation relates are distinct from those capable of being embodied by the ester component (1) and/or the fatty alcohol component (4) required according to the invention.

This limitation relates more particularly to waxes made up of complex mixtures which are notably described in Ullmann's Encyclopedia of Industrial Chemistry 2015, Wiley-VCH Verlag GmbH & Co. KGaA.

Such waxes may notably be natural, but may also be synthetic.

The term “natural” wax is intended to denote any wax which pre-exists in nature or which can be converted, extracted or purified from natural compounds which exist in nature.

Among natural waxes, mention may notably be made of waxes termed fossil waxes, including those of petroleum origin, such as ozokerite, pyropissite, macrocrystalline waxes, also known as paraffins—including crude or gatsch waxes, gatsch raffinates, de-oiled gatsch, soft waxes, semirefined waxes, filtered waxes, refined waxes—and microcrystalline waxes, termed microwaxes, including bright stock gatsch. The fossil waxes also contain lignite, also known as montan wax, or peat wax.

As natural waxes other than fossil waxes, mention may be made of animal and plant waxes.

As examples of plant waxes, mention may be made of carnauba wax, candelilla wax, ouricury wax, sugarcane wax, jojoba wax, Trithrinax campestris wax, raffia wax, alfalfa wax, wax extracted from Douglas fir, sisal wax, flax wax, cotton wax, Batavia dammar wax, cereal wax, tea wax, coffee wax, rice wax, palm wax, Japan wax, mixtures thereof and derivatives thereof.

As examples of animal waxes, mention may be made of beeswax, Ghedda wax, shellac, Chinese wax, lanolin, also known as wool wax, mixtures thereof and derivatives thereof.

These waxes are generally multicomponent. For example, natural beeswax is composed of approximately 70% of esters for the majority of monoesters (of fatty acid and of fatty alcohol), but also of hydroxy esters, of diesters and triesters and esters of sterols, and also of long-chain linear hydrocarbons, of free acids and of free alcohols. For obvious reasons, the weight proportion of their ingredients and their degree of purity are difficult to guarantee from one production batch to another.

The term “synthetic” wax is intended to denote waxes whose synthesis requires one or more chemical reactions performed by a human being.

Among the synthetic waxes, semisynthetic waxes and totally synthetic waxes may be distinguished. Synthetic waxes may be waxes obtained via a Fischer-Tropsch process, constituted for example of paraffins with a number of carbon atoms ranging from 20 to 50 or waxes of polyolefins, for example homopolymers or copolymers of ethylene, of propene or butene, or even longer-chain a-olefins. The latter can be obtained by thermomechanical degradation of polyethylene plastic, via the Ziegler process, via high-pressure processes, or else via processes catalyzed with metallocene species. These waxes may be crystallizable, partially crystallizable or amorphous. The abovementioned synthetic waxes are generally apolar and can be chemically treated to obtain polar waxes, for example via one or more of the following reactions: air oxidation, grafting, esterification, neutralization with metal soaps, amidation, direct copolymerizations or addition reactions.

In this case also, their composition may be constituted of a mixture of ingredients since the fatty-chain lengths are not well defined, thus forming a mixture of compounds having different fatty-chain lengths and for which it is difficult for manufacturers to guarantee perfect reproducibility from one production batch to another.

Consequently, the compositions according to the invention advantageously comprise less than 5% by weight, preferably less than 3% by weight of waxes, notably of multicomponent natural or synthetic waxes, relative to the total weight of the composition.

For the purposes of the invention, a multicomponent wax denotes a wax consisting of a mixture of several ingredients, either such as exists naturally like natural waxes, or such as is formed during the process of industrial synthesis of these materials.

In a particularly preferred embodiment of the invention, the composition is free of these waxes, notably multicomponent natural or synthetic waxes.

As specified above, the preferred texturing compounds according to the invention are by contrast and advantageously synthetic, single-component compounds, which are thus available in a form purified to more than 99%, like compound (1) required according to the invention.

b) Other Surfactants

The composition according to the invention may comprise surfactants other than that formed by the neutralization of a fatty acid having from 14 carbon atoms to less than 20 carbon atoms (2) with the base (3), as co-surfactants.

However, according to a preferred embodiment of the invention, the composition comprises less than 5.0% by weight, preferably less than 2.0% by weight of nonionic surfactants with an HLB, measured at 25° C., of greater than or equal to 7, relative to the total weight of the composition.

The term “HLB” (Hydrophilic Lipophilic Balance) is well known to those skilled in the art, and denotes the hydrophilic-lipophilic balance of a surfactant determined at 25° C. in the Griffin sense. The term “hydrophilic-lipophilic balance (HLB)” means the equilibrium between the size and the strength of the hydrophilic group and the size and the strength of the lipophilic group of the surfactant. The HLB value according to Griffin is defined in J. Soc. Cosm. Chem. 1954 (volume 5), pages 249-256.

In a particularly preferred embodiment of the invention, the composition is free of nonionic surfactants with an HLB, measured at 25° C., of greater than or equal to 7.

c) Hydrophilic Film-Forming Polymer(s)

A composition according to the invention may preferably comprise at least one hydrophilic film-forming polymer.

For the purposes of the present invention, the term “hydrophilic polymer” means a water-soluble polymer.

For the purposes of the present invention, the term “water-soluble polymer” refers to a polymer which, when introduced into water at a concentration equal to 1%, gives a macroscopically homogeneous solution whose light transmittance, at a wavelength equal to 500 nm, through a sample 1 cm thick, is at least 10%.

For the purposes of the present invention, the term “film-forming polymer” refers to a polymer that is capable, by itself or in the presence of an auxiliary film-forming agent, of forming a macroscopically continuous deposit, and preferably a cohesive deposit, and even better still a deposit whose the cohesion and mechanical properties are such that said deposit is isolable and can be manipulated individually, for example when said deposit is prepared by pouring onto a nonstick surface such as a Teflon-coated or silicone-coated surface.

For the purposes of the invention, a hydrophilic film-forming polymer that is particularly advantageous is a polyvinylpyrrolidone hydrophilic polymer.

Thus, preferably, a composition according to the invention comprises at least one polyvinylpyrrolidone hydrophilic polymer.

A polyvinylpyrrolidone hydrophilic polymer that is suitable for use in the invention may have a weight-average molecular mass, Mw, ranging from 1500 to 500 000 g/mol.

A composition according to the invention has a total solids content of (poly)vinylpyrrolidone hydrophilic polymer(s) of greater than or equal to 0.5% by weight, preferably greater than or equal to 1.0% by weight, more preferentially greater than or equal to 1.5% by weight, relative to the total weight of the composition.

The composition according to the invention preferably comprises from 0.1% to 15.0% by weight, preferably from 0.5% to 10.0% by weight, better still from 1.0% to 8.0% by weight of hydrophilic film-forming polymer(s), relative to the total weight of the composition.

Preferably, the (poly)vinylpyrrolidone hydrophilic polymer(s) in accordance with the invention are linear.

In particular, the (poly)vinylpyrrolidone hydrophilic polymer(s) in accordance with the invention are chosen from random polymers, block copolymers, and a mixture thereof. The term “block copolymer” means a polymer comprising at least two different blocks and preferably at least three different blocks.

The (poly)vinylpyrrolidone hydrophilic polymer(s) are chosen from:

(poly)vinylpyrrolidone homopolymers,

(poly)vinylpyrrolidone/(poly)vinyl acetate esters copolymers,

(poly)vinylpyrrolidone/(meth)acrylic copolymers, salts thereof, thereof and a mixture thereof.

As (poly)vinylpyrrolidone homopolymers, examples that may be mentioned include:

the polyvinylpyrrolidone (2500 g/mol) sold under the trade name Kollidon 17 PF by the company BASF,

the polyvinylpyrrolidone sold under the trade name Luviskol K 30 Powder by the company BASF or sold under the trade name PVP K 30L by the company ISP (Ashland),

the polyvinylpyrrolidone sold under the trade name PVP K 90 by the company ISP (Ashland).

As (poly)vinylpyrrolidone/(poly)vinyl acetate copolymers, mention may for example be made of the vinylpyrrolidone/vinyl acetate (60/40) copolymer sold under the trade name Luviskol VA 64 Powder by the company BASF.

d) Liquid Fatty Phase

A composition according to the invention generally comprises less than 5.0% by weight, preferably less than 2.0% by weight of volatile oil(s), relative to the total weight of the composition. In one particularly preferred embodiment of the invention, the composition is free of volatile oils.

The term “volatile oil” refers to an oil that can evaporate on contact with the skin in less than one hour, at room temperature (20° C.) and atmospheric pressure (760 mmHg). More specifically, a volatile oil has an evaporation rate ranging from 0.01 to 200 mg/cm²/min.

e) Dyestuff

A composition according to the invention, and notably those intended for makeup, generally comprises at least one dyestuff such as pulverulent materials, liposoluble dyes or water-soluble dyes.

The pulverulent dyestuffs may be chosen from pigments and nacres.

The pigments may be white or colored, mineral and/or organic, and coated or uncoated. Among the mineral pigments that may be mentioned are titanium dioxide, optionally surface-treated, zirconium oxide, zinc oxide or cerium oxide, and also iron oxide, chromium oxide, manganese violet, ultramarine blue, chromium hydrate and ferric blue. Among the organic pigments that may be mentioned are carbon black, pigments of D & C type and lakes based on cochineal carmine or on barium, strontium, calcium or aluminum.

The nacres may be chosen from white nacreous pigments such as mica coated with titanium or with bismuth oxychloride, colored nacreous pigments such as titanium mica with iron oxides, titanium mica notably with ferric blue or chromium oxide, titanium mica with an organic pigment of the abovementioned type, and also nacreous pigments based on bismuth oxychloride.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow and annatto.

Preferably, the composition according to the invention comprises a pulverulent dyestuff, preferably of pigment type, in particular metal oxides.

Preferably, a composition according to the invention also comprises from 2.0% to 25.0% by weight, preferably from 3.0% to 20.0% by weight and more particularly from 4.0% to 15.0% by total weight of dyestuff, relative to the total weight of the composition.

f) Cosmetic Active Agents

As cosmetic active agents that may be used in the compositions according to the invention, mention may be made notably of antioxidants, preserving agents, fragrances, neutralizers, cosmetic active agents, for instance emollients, vitamins and screening agents, in particular sunscreens, and mixtures thereof.

These additives may be present in the composition in a content ranging from 0.01% to 15.0% by weight, relative to the total weight of the composition.

Needless to say, a person skilled in the art will take care to select the optional additional additives and/or the amount thereof such that the advantageous properties of the composition according to the invention are not, or are not substantially, adversely affected by the envisaged addition.

### Physical Characteristics

a) Solids Content

The composition according to the invention advantageously has a solids content of at least 42.0% by weight, preferably of at least 44.0% by weight or even from 45.0% to 60.0% by weight, relative to the total weight of the composition.

For the purposes of the present invention, the “solids content” denotes the content of nonvolatile matter.

The amount of solids content (abbreviated as SC) of a composition according to the invention is measured using a Halogen Moisture Analyzer HR 73 commercial halogen desiccator from Mettler Toledo. The measurement is performed on the basis of the weight loss of a sample dried by halogen heating, and thus represents the percentage of residual matter once the water and the volatile matter have evaporated off.

This technique is fully described in the machine documentation supplied by Mettler Toledo.

The measuring protocol is as follows:

Approximately 2 g of the composition, referred to hereinbelow as the sample, are spread out on a metal crucible, which is placed in the halogen desiccator mentioned above. The sample is then subjected to a temperature of 105° C. until a constant weight is obtained. The wet mass of the sample, corresponding to its initial mass, and the dry mass of the sample, corresponding to its mass after halogen heating, are measured using a precision balance.

The experimental error associated with the measurement is of the order of plus or minus 2%.

The solids content is calculated in the following manner:

$$Solids content ( expressed as \% by weight ) = 100 \times \frac{Dry mass}{Wet mass} \qquad [ Math 1 ]$$

b) Viscosity

A composition according to the invention is advantageously creamy at an ambient temperature of 20° C.

It is characterized by a viscosity of less than 40 Pa·s, or even preferably less than 35 Pa·s, or even less than 30 Pa·s, measured at an ambient temperature of 20° C. using a Rheomat RM100® viscometer.

Preferably, the viscosity of the compositions according to the invention ranges from 2.0 to 40.0 Pa·s, or even preferably from 2.5 to 35.0 Pa·s, more particularly from 3.0 to 30.0 Pa·s, measured at the ambient temperature of 20° C. using a Rheomat RM100® viscometer.

Such a viscosity is particularly advantageous since it is the most suitable for the device for applying mascara and since it enables easy use for the consumer for a charging result.

The composition may be manufactured via the known processes generally used in the cosmetics field.

The composition used according to the invention may be a makeup composition, a makeup base, notably for keratin fibers, or base coat, a composition to be applied onto makeup, also known as topcoat, or else a composition for treating keratin fibers.

More especially, the composition according to the invention is a mascara.

Such compositions are notably prepared according to the general knowledge of those skilled in the art.

The expressions “between . . . and . . . ” and “ranging from . . . to . . . ” should be understood as being inclusive of the limits, unless otherwise specified.

In the description and the examples, the percentages are weight percentages, unless otherwise indicated. The percentages are thus expressed on a weight basis relative to the total weight of the composition. The ingredients are mixed in the order and under the conditions which are readily determined by a person skilled in the art.

The invention will now be described by means of examples which are presented for purely illustrative purposes and should not be interpreted as examples that limit the invention.

### EXAMPLE

Compositions of mascara type in accordance with the invention (Compositions 1 to 5) and a composition not in accordance with the invention (Composition 6) were prepared as described below.

Preparation of Phase A

The starting materials were carefully weighed out beforehand using a balance (precision=0.01 g). The ingredients of phase A were melted in a jacketed heating pan in which circulates an oil whose temperature is controlled by means of a thermostatically-controlled oil bath. The nominal temperature was set at 90° C. After total melting, the pigment was introduced with stirring using a Rayneri blender. Stirring was maintained until a homogeneous preparation was obtained.

Preparation of Phase B

The water was preheated in an electric kettle to 95° C. The preserving agents and the base were introduced into the water in a beaker at a temperature of 80° C. with stirring using a Rayneri blender.

Emulsification of Phases A and B

Phase B was poured into phase A with stirring for 5 minutes at 90° C. using a Rayneri blender. Phase A+B was then cooled to room temperature with stirring.

Table 1 below details the respective compositions thereof.

Each mascara thus obtained was transferred into a closed container to prevent it from drying out on contact with air. The stability of the samples (the state of dispersion of the fatty substances and pigments was evaluated using a light microscope) and the viscosity were examined.

TABLE 1

| | | | | | | | | 6 not in |
|---|---|---|---|---|---|---|---|---|
| | | | | | | | | accordance |
| | INCI Name | Commercial reference | 1 | 2 | 3 | 4 | 5 | with invention |
| A | CETEARYL ALCOHOL | Lanette O OR/MB from | 4.00 | 4.00 | 4.00 | 4.00 | 4.00 | 4.00 |
| | (C₁₆₋C₁₈₋) (compound (4)) | BASF | | | | | | |
| | BEHENYL BEHENATE | Kester wax K-72 from | 15.00 | 15.00 | 15.00 | 15.00 | / | / |
| | (C₄₄) (compound (1)) | Koster Keunen | | | | | | |
| | TRIBEHENIN | Synchrowax HRC-PA- | / | / | / | / | 15.00 | 21.00 |
| | (compound (1)) | (MH) from Croda | | | | | | |
| | C₁₀₋₃₀ POLY(ALKYL | Intelimer IPA 13-6 | 6.00 | 6.00 | 6.00 | 6.00 | 6.00 | / |
| | ACRYLATE) (C₂₂) | Polymer from Air | | | | | | |
| | (compound (5)) | Products and Chemicals | | | | | | |
| | STEARIC ACID | Stearic acid 1850 from | 6.00 | 6.00 | 6.00 | 6.00 | 6.00 | 6.00 |
| | (compound (2)) | Southern Acids | | | | | | |
| | IRON OXIDES | Sunpuro Black Iron oxide | 8.00 | 8.00 | 8.00 | 8.00 | 8.00 | 8.00 |
| | | C33-7001 from Sun | | | | | | |
| | POLYVINYL- | Polyvinylpyrrolydone K | 2.00 | 2.00 | 2.00 | 2.00 | 2.00 | 2.00 |
| | PYRROLYDONE | 30 Powder from BASF | | | | | | |
| | C₁₈-C₃₆ ACID | DUB TGI 24 from | 5.00 | 10.00 | / | / | 5.00 | 5.00 |
| | TRIGYLCERIDE | Stéarinerie Dubois | | | | | | |
| | (compound (6)) | | | | | | | |
| | POLYBUTENE | Indopol H 100 from Ineos | / | / | 5.00 | / | | |
| | (compound (6)) | Indopol H 300 from Ineos | / | / | / | 10.00 | | |
| B | PHENOXYETHANOL | Protectol PE CO from | 0.60 | 0.60 | 0.60 | 0.60 | 0.60 | 0.60 |
| | | BASF | | | | | | |
| | CHLORPHENESIN | Cosvat L from Kraeber | 0.30 | 0.30 | 0.30 | 0.30 | 0.30 | 0.30 |
| | CAPRYYL GLYCOL | 199602 Hydrolite CG | 0.80 | 0.80 | 0.80 | 0.80 | 0.80 | 0.80 |
| | | from Symrise | | | | | | |
| | AMINOMETHYL | AMPD ULTRA PC from | 1 | 1 | 1 | 1 | 1 | 1 |
| | PROPANEDIOL | Angus (Dow Chemicals) | | | | | | |
| | (compound (3)) | | | | | | | |
| | WATER (compound (7)) | | qs | qs | qs | qs | qs | qs |
| | Viscosity at 24 H (in Pa · s) | | 8 | 20.5 | 2.4 | 12.3 | 8.5 | Very thick |
| | | | | | | | | formulation |

| | | | | | | |
|---|---|---|---|---|---|---|
| Viscosity after 2 months at 45° C.(in Pa · s) | 7.8 | 14.2 | 2.6 | 12.0 | 7.1 | — |

Compositions 1 to 5 have performance qualities suitable to allow them to be used for eyelash care and/or makeup purposes. Their textures are pleasant on application. They have a long playtime. Thus, when they are applied to eyelashes, the eyelash separation is preserved even after a large number of brush strokes.

On the other hand, composition 6 not in accordance with the invention has a texture that is too rigid, incompatible with spreading properties as desired by users. Furthermore, such a formulation does not allow a homogeneous composition to be obtained.

The stability of the compositions was examined after all the preceding compositions spent a residence time of two months at 45° C. The texture of the sample which resided at 45° C. was then compared with that of the sample which remained at room temperature.

A stable composition is a composition which conserves its texture and its homogeneity and which remains pleasant to apply. Conversely, a composition is termed unstable if its texture becomes much thicker, if phase separation appears or if the composition is no longer pleasant on application.

The results obtained are all reported in Table 2 below:

TABLE 2

| | | | | | | Composition |
|---|---|---|---|---|---|---|
| | | | | | | not in |
| | Composition | Composition | Composition | Composition | Composition | accordance |
| | according to | according to | according to | according to | according to | with the |
| | the invention | the invention | the invention | the invention | the invention | invention |
| | 1 | 2 | 3 | 4 | 5 | 6 |
| Appearance | Creamy | Creamy | Creamy | Creamy | Creamy | Too rigid |
| to the | texture | texture | texture | texture | texture | texture |
| naked eye | | | | | | |
| Microscope | Fine and | Fine and | Fine and | Fine and | Fine and | / |
| | homogenous | homogenous | homogenous | homogenous | homogenous | |
| | emulsion | emulsion | emulsion | emulsion | emulsion | |
| Stability | Stable | Stable | Stable | Stable | Stable | Unstable |

Only composition 6, not having the features of the invention, proves to be unstable.

1. A composition, for caring for and/or making up keratin materials, comprising:
(1) at least one ester chosen from:
(i) linear fatty acid monoesters of formula (I):
R₁—O—R₂ (I)
in which R₁ and R₂ are linear and saturated and have, independently of each other, a number of carbon atoms greater than or equal to 20, with R₁ representing an acyl radical, and R₂ representing an alkyl radical, and
(ii) fatty acid esters of polyol, said fatty acid(s) having a number of carbon atoms greater than or equal to 20,
(2) at least one fatty acid having from 14 carbon atoms to less than 20 carbon atoms,
(3) at least one base that is capable of at least partially neutralizing said fatty acid (2),
(4) at least one fatty alcohol preferably chosen from C₁₄-C₃₀ fatty alcohols,
(5) at least one semicrystalline polymer,
(6) at least one nonvolatile hydrocarbon-based oil, and
(7) water.

2. The composition as claimed in claim 1, containing less than 5% by weight of waxes relative to the total weight of said composition.

3. The composition as claimed in claim 1, comprising at least 5.0% by weight of ester (1) relative to the total weight of said composition.

4. The composition as claimed in claim 1, in which said ester (1) is at least one fatty acid ester of polyol (ii) chosen from the fatty acid esters of glycerol of formula (II): 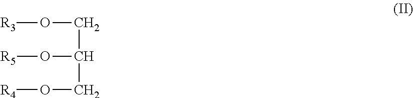
in which R₃, R₄ and R₅ may represent, independently of each other, a hydrogen atom H or a saturated or unsaturated, linear or branched acyl radical having a number of carbon atoms greater than or equal to 20, at least one from among R₃, R₄ and/or R₅ representing a saturated or unsaturated, linear or branched acyl radical having a number of carbon atoms greater than or equal to 20.

5. (canceled)

6. The composition as claimed in claim 4, in which said fatty acid ester of polyol (ii) is chosen from a fatty acid triester of glycerol and a mixture of fatty acid monoester, diester and triester of glycerol.

7. The composition as claimed in claim 1, in which said ester (1) is at least one linear fatty acid monoester of formula (I) in which R₁ and R₂ are, respectively, acyl and alkyl radicals having the same number of carbon atoms.

8. (canceled)

9. The composition as claimed in claim 7, in which the linear fatty acid monoester (i) is chosen from arachidyl arachidate and behenyl behenate.

10. The composition as claimed in claim 1, in which the fatty acid(s) (2) are chosen from linear fatty acids and saturated fatty acids, and mixtures thereof.

11. The composition as claimed in claim 1, in which the fatty acid(s) (2) are chosen from stearic acid and palmitic acid, and mixtures thereof.

12. The composition as claimed in claim 1, comprising at least 3.0% by weight of fatty acid (2) relative to the total weight of said composition.

13. The composition as claimed in claim 1, in which the base (3) is at least one organic base.

14. The composition as claimed in claim 1, comprising at least 0.1% by weight of base(s) (3) relative to the total weight of said composition.

15. The composition as claimed in claim 1, comprising at least one fatty alcohol (4) chosen from cetyl alcohol (C₁₆), stearyl alcohol (C₁₈) and mixtures thereof.

16. The composition as claimed in claim 1, comprising at least from 1.0% to 20.0% by weight of fatty alcohol(s) (4), relative to the total weight of the composition.

17. The composition as claimed in claim 1, in which the semicrystalline polymer(s) (5) are derived from a monomer containing a crystallizable chain chosen from saturated C₁₀ to C₃₀ alkyl (meth)acrylates and mixtures thereof.

18. The composition as claimed in claim 1, in which the semicrystalline polymer(s) (5) are at least one poly(behenyl acrylate).

19. The composition as claimed in claim 1, comprising at least 2.0% by weight of semicrystalline polymer(s) (5), relative to the total weight of the composition.

20. The composition as claimed in claim 1, comprising at least one nonvolatile hydrocarbon-based oil (6) which has a molar mass ranging from 650 g.mol⁻¹ to 7500 g.mol⁻¹.

21. The composition as claimed in claim 1, comprising between 0.1% and 20% by weight of nonvolatile oil(s) (6) relative to the total weight of said composition.

22. The composition as claimed in claim 1, the solids content of which is at least 42.0% by weight relative to the total weight of said composition.

23. The composition as claimed in claim 1, comprising at least 30% by weight of water (7), relative to the total weight of said composition.

24. The composition as claimed in claim 1, comprising less than 5.0% by weight of volatile oil(s), relative to the total weight of said composition.

25. The composition as claimed in claim 1, comprising less than 5.0% by weight of nonionic surfactants with an HLB, measured at 25° C., of greater than or equal to 7, relative to the total weight of said composition.

26. The composition as claimed in claim 1, comprising at least one polyvinylpyrrolidone hydrophilic polymer.

27. The composition as claimed in claim 1, comprising at least one pulverulent dyestuff.

28. The composition as claimed in claim 1, also comprising from 2.0% to 25.0% by weight of dyestuff, relative to the total weight of said composition.

29. A process for caring for and/or making up keratin materials, applying to said keratin materials, a composition as claimed in claim 1.
